(19) United States
(12) Patent Application Publication
(10) Pub. No.: US 2025/0213451 A1
(43) Pub. Date: Jul. 3, 2025

(54) AN ORAL CARE COMPOSITION COMPRISING A GLYCOLIPID AND A MILD SURFACTANT

(71) Applicant: BASF SE, Ludwigshafen (DE)

(72) Inventors: Hua Zhou, Shanghai (CN); Yu Ting Xu, Shanghai (CN); Yi Yao Wang, Shanghai (CN); Ben Chuan Zhu, Shanghai (CN); Annette Mehling, Düsseldorf-Holthausen (DE)

(21) Appl. No.: 18/688,943

(22) PCT Filed: Aug. 29, 2022
(86) PCT No.: PCT/EP2022/073960

§ 371 Date: Mar. 4, 2024

(30) Foreign Application Priority Data
Sep. 9, 2021 (WO) PCT/CN2021/117442

(51) Int. Cl. A61K 8/60; A61K 8/44; A61K 8/49; A61Q 11/00

(52) U.S. Cl. CPC A61K 8/604; A61K 8/44; A61K 8/4973; A61K 8/602; A61Q 11/00

(57) ABSTRACT

The present invention relates to an oral care composition comprising at least one glycolipid and at least one surfactant selected from the group consisting of alkyl polyglycoside surfactant, carboxylated alkyl polyglycoside surfactant and N-acyl amino acid surfactant. The present invention also provides a method for removing and/or preventing dental plaque.

### TECHNICAL AREA

The present invention provides an oral care composition comprising a glycolipid, and a surfactant selected from the group consisting of alkyl polyglycoside surfactant, carboxylated alkyl polyglycoside surfactant, and N-acyl amino acid surfactant. The present invention further provides a method for removing plaque present on dental surfaces or preventing plaque.

### BACKGROUND ART

Oral compositions such as toothpastes, gels and mouth washes are designed to loosen and remove plaque in conjunction with a regular toothbrushing regimen. Dental plaque is present to some degree, in the form of a film, on virtually all dental surfaces. It is a byproduct of microbial growth, and comprises a dense microbial layer consisting of a mass of microorganisms embedded in a polysaccharide matrix. Plaque itself adheres firmly to dental surfaces and is very difficult to remove even through a rigorous brushing regimen. Moreover, plaque rapidly reforms on the tooth surface after it is removed. Plaque may form on any part of the tooth surface, and is found particularly at the gingival margin, in cracks in the enamel, and on the surface of dental calculus. Dental plaque is a mixed matrix of bacteria, epithelial cells, leukocytes, macrophages and other oral exudate. Bacteria comprise approximately three-quarters of the plaque matrix. Any given sample of dental plaque could contain as many 400 different varieties of microorganisms. This mix includes both aerobic and anaerobic bacteria, fungi and protozoa. Viruses have also been found in samples of dental plaque. The danger and consequence associated with the formation of plaque on the teeth will be the tendency of plaque to build up and eventually produce gingivitis, periodontitis and other types of periodontal disease, as well as dental caries and dental calculus.

The failure to retard or stop the proliferation of plaque is detrimental to oral health. Plaque formation leads to dental caries, gingival inflammation, periodontal disease and ultimately tooth loss. The present inventors recognize these problems and have developed a composition which can work more effectively on oral biofilm inhibition and prevent the formation of plaque onto the teeth surface.

Another concern for oral care composition is to have an efficient surfactant which may effectively be used to inhibit the formation of dental plaque. Most of anionic surfactants used in oral care or personal care formulation generally exhibit superior cleansing and foaming properties, but these anionic surfactants tend to cause irritation to human oral mucosa in levels typically used. In order to produce more mild oral care compositions, it is desirable to replace some of the anionic surfactants by mild surfactants without or with less irritation.

The use of a variety of agents to clean the oral cavity and reduce plaque has been recognized for some time. Examples include U.S. Pat. No. 5,320,831 disclosing an oral care composition containing an enzyme, a surfactant, a chelating agent and a fluoride ion source. U.S. Pat. No. 2002/0081267 disclosing an anti-plaque emulsion comprising a surfactant, emulsifier and triclosan.

While the prior art discloses the use of various oral compositions for combating plaque, there is still a need for additional formulations which provide improved performance in combating oral disease along with increased user acceptance without compromising the mildness and safety properties of the oral care formulation.

### SUMMARY OF THE INVENTION

In one aspect, the subject of the present invention is in relation to an oral care composition comprising at least
(i) a glycolipid, and
(ii) a surfactant selected from the group consisting of alkyl polyglycoside surfactant, carboxylated alkyl polyglycoside surfactant, N-acyl amino acid surfactant, and a mixture thereof.
wherein the glycolipid is preferably sophorolipid.

In a further aspect, wherein the sophorolipid is a mixture of acidic form and lactone form; preferably about 10 to about 90 wt. %, more preferably about 20 to about 60 wt. %, still more preferably about 25 wt. % to 40 wt. % of the sophorolipid being in acidic form and the remainder of the sophorolipid being in the lactone form.

In another aspect, the present invention relates to a method for removing plaque present on dental surfaces or preventing plaque formation, comprising the application of a safe and effective amount of the oral care composition of the present invention to the teeth and other oral surfaces.

In still another aspect, the present invention relates to use of the oral care composition of the present invention for removing plaque present on dental surfaces or preventing plaque.

Unpredictably, the applicant has found that the abovementioned aim can be achieved using a glycolipid and at least one surfactant selected from the group consisting of alkyl polyglycoside surfactant, carboxylated alkyl polyglycoside surfactant, N-acyl amino acid surfactant and a mixture thereof. It is extremely surprising that synergistic reduction or inhibition of plaque compared to the separate use of either compound alone has been found, while also increasing mildness.

### DETAILED DESCRIPTION OF THE INVENTION

While the specification concludes with claims that particularly point out and distinctly claim the invention, it is believed the present invention will be better understood from the following description.

Throughout the description, including the claims, the term “comprising one” or “comprising a” should be understood as being synonymous with the term “comprising at least one”, unless otherwise specified, and “between” should be understood as being inclusive of the limits.

The articles “a”, “an” and “the” are used to refer to one or to more than one (i.e., to at least one) of the grammatical object of the article.

The term “and/or” includes the meanings “and”, “or” and also all the other possible combinations of the elements connected to this term.

The term “active compound” and/or “active ingredient” used herein, refers to the ingredient or ingredients that improve or maintain the oral health.

The term “dentifrice” used herein, refers to paste, gel, powder, tablets, or liquid formulations, unless otherwise specified, that are used to clean the surfaces of the oral cavity. The term “teeth” as used herein refers to natural teeth as well as artificial teeth or dental prosthesis.

The term “alkyl” as used herein refers to a saturated hydrocarbon radical, which may have for example 1 to 6 (“C₁-C₆-alkyl”), 1 to 4 (“C₁-C₄-alkyl”) or 1 to 3 (“C₁-C₃-alkyl”) carbon atoms. Examples of C₁-C₃-alkyl include methyl, ethyl, propyl and isopropyl. Examples of C₁-C₄-Alkyl additionally include n-butyl, 1-methylpropyl (sec-butyl), 2-methylpropyl (isobutyl) and 1,1-dimethylethyl (tert-butyl). Examples of C₁-C₆-alkyl further includes n-pentyl, 1-methylbutyl, 1-ethylpropyl, neo-pentyl, n-hexyl, 1-methylpentyl, 2-methylpentyl, 1-ethylbutyl, 2-ethylbutyl, etc.

The term “alkylene” as used herein refers to a divalent alkanediyl radical, which may have for example 1 to 6 carbon atoms (“C₁-C₆-alkylene”), 1 to 4 carbon atoms (“C₁-C₄-alkylene”) or 1 to 3 carbon atoms (“C₁-C₃-alkylene”). Examples of C₁-C₄-alkylene include, but are not limited to methylene, 1,2-ethylene, 1,3-propylene and 1,4-butylene. Examples of C₁-C₆-alkylene additionally include 1,5-pentylene, 1,6-hexylene, and isomers thereof.

The term “hydrocarbyl” as used herein refers to a radical of a saturated or unsaturated hydrocarbon, and may include, but are not limited to “alkyl” and “alkenyl”.

The term “acyl” as used herein refers to a straight-chain or branched hydrocarbyl that is bonded to the remainder of the compound directly via carbonyl, which may be represented by “hydrocarbyl —C(O)—”.

The term “oral care composition” used herein, refers to a product which in the ordinary course of usage is not intentionally swallowed for purpose of systemic administration of systemic administration of particular therapeutic agents, but is rather retained in the mouth for a time sufficient to contact substantially all of the dental surfaces and/or oral tissues for purposes of oral activity.

By “safe and effective amount,” as used herein, means a sufficient amount to reduce plaque/gingivitis without harming the tissues and structures of the oral cavity.

By the term “suitable oral carrier,” as used herein, means a suitable vehicle which can be used to apply the present compositions to the oral cavity in a safe and effective manner.

It should be noted that in specifying any range of concentration, weight ratio or amount, any particular upper concentration, weight ratio or amount can be associated with any particular lower concentration, weight ratio or amount, respectively. Unless otherwise specified, all percentages are by weight.

The present invention is related to an oral care composition comprising at least
(i) a glycolipid, and
(ii) a surfactant selected from the group consisting of alkyl polyglycoside surfactant, carboxylated alkyl polyglycoside surfactant, N-acyl amino acid surfactant and a mixture thereof,
   wherein the glycolipid is preferably sophorolipid.

The oral care composition of the present invention comprising a glycolipid and at least one alkyl polyglycoside surfactant, carboxylated alkyl polyglycoside surfactant, N-acyl amino acid surfactant or a mixture thereof, demonstrates synergistic reduction or inhibition of plaque compared to the separate use of either compound alone, while also increasing mildness with lower oral irritation level.

### Glycolipid

The oral care composition of the present invention comprises at least one glycolipid.

Glycolipid is widely used as biosurfactant in personal care formulations. Biosurfactant is understood to be substances that are formed by microorganisms and are often expelled from the cell. Like classic surfactants, biosurfactant is surface-active substance that reduce the surface tension of liquids and thereby promote the mixing of aqueous (hydrophilic) and water-repellent (hydrophobic) phases. Biosurfactant can be produced under gentle production conditions that require little energy. They are generally easily biodegradable and are very environmentally friendly. Moreover, they are not toxic, nor are any toxic byproducts produced during the production thereof. Carbohydrate, in particular sugar, e.g. glucose, and/or lipophobic carbon sources such as fats, oils, partial glycerides, fatty acids, fatty alcohols, long-chain saturated or unsaturated hydrocarbons, are used as raw materials for the microbial production of said biosurfactant. According to the present invention, the biosurfactant is preferably biosurfactant produced by fermentation.

Glycolipids that can be used in the present invention are compounds in which one or more monosaccharide units are glycosidically bonded to a lipid moiety. Examples of glycolipids that can be used according to the invention are sophorolipids, rhamnolipids, cellobioselipids, mannosyl erythritol lipids, trehalose lipids and biochemical modification thereof. In some preferred embodiments, the oral care composition of the present invention comprising a sophorolipid.

Sophorolipids are produced by fermentation using yeasts such as Starmerella(Candida) Bombicola (also known as Torulopsis bombicola), Yarrowia lipolytica, Candida apicola (Torulopsis apicola) and Candida bogoriensis, by growing said yeasts on sugars, hydrocarbons, plant oils or mixture thereof. Sophorolipids have the following formulae (a) (lactone form) and (b) (free acid), the two forms typically being provided in a mixture,

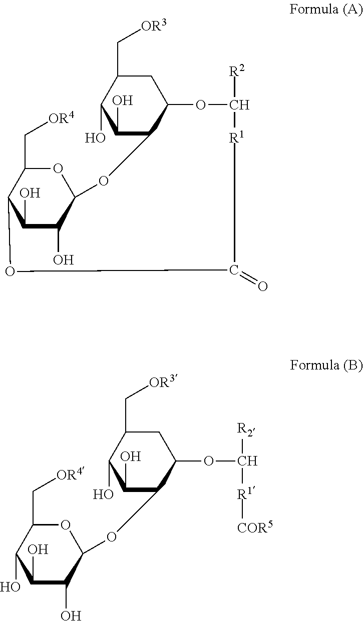

Where R¹ and R^{1′} independently represent saturated hydrocarbon chains or single or multiple, in particular single, unsaturated hydrocarbon chains having 8 to 20, in particular 12 to 18, carbon atoms, more preferably 14 to 18 carbon atoms, which can be linear or branched and can include one or more hydroxy groups, R² and R^{2′} independently represent a hydrogen atom or an methyl group. Sophorolipid in which R¹ and R^{1′} are single, unsaturated, linear hydrocarbon chains having 15 carbon atoms are preferred. It is also preferred for R² and R^{2′} to represent a methyl group or a hydrogen atom or a saturated alkyl functional group or a single or multiple, in particular single, unsaturated alkyl functional group having 1 to 9 carbon atoms, which can be linear or branched and can include one or more hydroxy groups, and R³, R^{3′}, R⁴ and R^{4′} independently represent a hydrogen atom or an acetyl group.

According to the present invention, sophorolipids in which the acidic form and the lactone form in a mixture are preferred, preferably about 10 to about 90 wt. %, more preferably about 20 to about 60 wt. %, still more preferably about 25 wt. % to 40 wt. % of the sophorolipid being in acidic form and the remainder of the sophorolipid being in the lactone form.

Sophorolipids being suitable used for the present invention can be obtained commercially, for example under the trade name BioToLife™ from BASF. The sophorolipid supplied by BASF, about 30 wt. % is present in the free acid form, in a mixture with the lactone form.

According to any one of the invention embodiments, the oral care composition of the invention comprises from more than 0 to 10% of sophorolipid relative to the total weight of the composition, preferably from 0.1% to 5%, more preferably from 0.15% to 3.5%, or from 0.2% to 2%, or from 0.25% to 1.0%

### Alkyl Polyglycoside Surfactant

The oral care composition of the present invention comprises a glycolipid and at least one surfactant selected from the group consisting of alkyl polyglycoside surfactant, carboxylated alkyl polyglycoside surfactant, N-acyl amino acid surfactant and a mixture thereof, wherein the glycolipid is preferably sophorolipid.

In some embodiments, the oral care composition of the present invention comprises a sophorolipid and an alkyl polyglycoside surfactant having a formula (I):

R₁—O—(R₂—O)ₐ—(Z)_{b}—H (I)
Wherein R₁ is a straight chain or branched C₆-C₂₄-hydrocarbyl,
   R₂ is a straight chain C₂-C₄-alkylene,
   a is a number in the range of 0 to 10, preferably 0 to 4,
   Z is a residue of a reducing sugar having 5 to 6 carbons, and
   b is a number in the range of 1 to 5, preferably 1 to 2 and any combinations thereof.

Alkyl polyglycosides can be produced by any well-known method to person skilled in the art, for example, by reacting a suitable carbohydrate with a fatty alcohol.

In above formula (I), R₁ is preferably a straight chain or branched C₈-C₁₈-alkyl, C₁₀-C₁₆-alkyl or C₁₂-C₁₄ alkyl for example n-octyl, n-nonyl, n-decyl, n-undecyl, n-dodecyl, n-tridecyl, n-tetradecyl, n-pentadecyl, n-hexadecyl, heptadecyl or octadecyl. Additionally, or alternatively, Z is a residue of a reducing sugar selected from glucose, fructose, galactose, xylose or arabinose, preferably glucose.

Preferably, in above formula (I), a is 0 and b is 1. Particularly, notable alkyl polyglycoside for the present invention are C₈-C₁₈ alkyl polyglycoside, for example decyl glucoside and lauryl glucoside, cetearyl glucoside, stearyl glucoside, cocyl glucoside, isostearyl glucoside, oleyl glucoside.

### Carboxylated Alkyl Polyglycoside Surfactant

The oral care composition of the present invention comprises a glycolipid at least one surfactant selected from the group consisting of alkyl polyglycoside surfactant, carboxylated alkyl polyglycoside surfactant, N-acyl amino acid surfactant and a mixture thereof, wherein the glycolipid is sophorolipid.

In some embodiments, the oral care composition of the present invention comprises a sophorolipid and a carboxylated alkyl polyglycoside surfactant having a formula (II):

R₃—O—(R₄—O)ₘ—(S)ₙ—CH₂COO⁻X⁺ (II)
Wherein R₃ is a straight chain or branched C₆-C₂₄-hydrocarbyl,
   R₄ is straight chain C₂-C₄-alkylene,
   m is a number in the range of 0 to 10, preferably 0 to 4,
   S is a residue of a reducing sugar having 5 to 6 carbons, and
   n is a number in the range of 1 to 5, preferably 1 to 2, and
   X is an alkali metal ion, and
any combinations thereof.

In above formula (II), R₃ is preferably a straight chain or branched C₈-C₁₈ alkyl, preferably straight chain C₁₀-C₁₆ or C₁₂-C₁₄ alkyl, for example n-octyl, n-nonyl, n-decyl, n-undecyl, n-dodecyl, n-tridecyl, n-tetradecyl, n-pentadecyl, n-hexadecyl, heptadecyl or octadecyl, more preferably n-dodecyl (lauryl). Additionally, or alternatively, S is a residue of a reducing sugar selected from glucose, fructose, galactose, xylose or arabinose, preferably glucose.

More particularly preferred carboxylated alkyl polyglycoside and the method of preparation of the carboxylated alkyl polyglycoside can be found in U.S. Pat. No. 6,248,792 by reference.

The carboxylated alkyl polyglycoside surfactants possess anionic properties. They provide superior levels of stable foam and act as viscosity builder when used in various types of detergent compositions.

A variety of carboxylated alkyl polyglycoside surfactants can be used in the composition of the present invention. The carboxylated alkyl polyglycosides can be made by such methods as the reaction of an alkyl polyglycoside with an alpha- or 2-halocarboxylic acid such as 2-chloroacetic acid, or by the reaction of an alkyl polyglycoside with an alpha, beta-unsaturated carboxylic acid such as acrylic acid or methacrylic acid, or by the reaction of an alkyl polyglycoside with a cyclic carboxylic acid anhydride such as succinic anhydride or maleic anhydride. The carboxylated alkyl polyglycoside can be therefore the reaction product of an alkyl polyglycoside with an alpha- or 2-halocarboxylic acid; the reaction product of an alkyl polyglycoside with an alpha, beta-unsaturated carboxylic acid; or the reaction product of an alkyl polyglycoside with a cyclic carboxylic acid anhydride.

Particularly preferred carboxylated alkyl polyglycoside surfactants are those of formula (II) where R₃ is a straight chain or branched C₈-C₁₆ hydrocarbyl, S is a residue of a reducing sugar selected from glucose, fructose, galactose, xylose or arabinose glucose residue, preferably glucose, n is about 1.4 to 2, preferably 1.4 to 1.6, more preferably 1.55, m is zero. Preferably, in above formula (II), m is 0, and n is 1.

In some preferred embodiments of the present invention, the oral care composition comprises a mixture of an alkyl polyglycoside surfactant and a carboxylated alkyl polyglycoside surfactant.

### N-Acyl Amino Acid Surfactant

The oral care composition of the present invention comprises a glycolipid and at least one surfactant selected from the group consisting of alkyl polyglycoside surfactant, carboxylated alkyl polyglycoside surfactant, N-acyl amino acid surfactant and a mixture thereof, wherein the glycolipid is preferably sophorolipid.

In some embodiments, the oral care composition of the present invention comprises a sophorolipid and at least one N-acyl amino acid surfactant. The N-acyl amino acid surfactant to be used in the present invention is N-long-chain acyl amino acids or their salts. The long-chain acryl group constitutes the N-long chain acyl acidic amino acid, a linear or branched acyl group having 6 to 20 carbon atoms, and the hydrocarbon chain may be either saturated or unsaturated. When the acyl group is unsaturated, two or more unsaturated bonds may be contained, and the unsaturated bonds may be either conjugated or not conjugated. Examples of the acyl group include, for example, acyl groups that can be derived from caproic acid, caprylic acid, capric acid, lauric acid, myristic acid, palmitic acid, stearic acid, behenic acid, linolic acid, linoleic acid, oleic acid, isostearic acid, 2-ethylhexanioic acid, coconut oil fatty acid, tallow fatty acid, hydrogenated tallow fatty acid and the like. Among them, acyl groups having 8 to 18 carbon atoms, or 10 to 16 carbon atoms are more preferred, and examples thereof include capryloyl group, caprinoyl group, lauroyl group, myristyl group, palmitoyl group, stearoyl group, coconut oil fatty acid acyl group (cocoyl group), hydrogenated tallow fatty acid acyl group, palm kernel oil fatty acid acyl group and the like. In the specification, a mixed acyl group means a mixture of acyl groups with different lengths of chains. Examples of the acidic amino acids constituting the long-chain acyl acidic amino acids include taurine, sarcosine, glycine, serine, alanine, lysine, arginine, proline, threonine, valine, isoleucine, histidine, phenylalanine, preferably glutamic acid, sarcosine and taurine, among them, glutamic acid and taurine are preferred in the present invention. Types of salts of the N-long chain acyl acidic amino acids are not particularly limited. Examples of the salts include sodium salt, magnesium salt, potassium salt, ammonium salt, diethanolamine salt, triethanolamine salt, arginine salt, lysine salt and the like.

Examples of the N-long-chain acyl acidic amino acids and salts thereof include N-long chain acyl-glutamic acids and salts thereof such as N-cocoyl glutamic acids and salts thereof, N-lauroylglutamic acid and salts thereof, N-stearoylglutamic acid and salts thereof, N-myristoylglutamic acid and salts thereof, N-palmitoylglutamic acid and salts thereof and N-oleoylglutamic acid and salts thereof; N-long chain acyl taurine and salts thereof such as N-cocoyl taurine and salts the thereof, N-stearoyl taurine and salts thereof, N-myristoyl taurine and salts thereof, N-palmitoyl taurine and salts thereof and N-oleoyl taurine and salts thereof and N-lauroyl taurine or salts thereof; N-long chain acyl sarcosine and salts thereof such as N-cocoyl sarcocine and salts the thereof, N-stearoyl sarcosine and salts thereof and N-lauroyl sarcosine or salts thereof and the like. Among them, particularly preferred examples include N-cocoyl glutamic acids and salts thereof and N-lauroylglutamic acid and salts thereof. Among them, particularly preferred examples include N-coconut oil fatty acid acyl glutamic acids and salts thereof and N-lauroylglutamic acid and salts thereof. Two or more kinds of N-long chain acyl acidic amino acids or salts thereof may be used in combination.

The total amount of the surfactants comprising at least one alkyl polyglycoside, carboxylated alkyl polyglycoside or N-acyl amino acid surfactant is from more than 0 to 5% based on the total weight of the composition, preferably from 0.1% to 4%, or from 0.15% to 3.5%, or from 0.2% to 3%.

In some embodiments of the present invention, the oral care composition comprises a mixture of alkyl polyglycoside and carboxylated alkylpolyglycoside, preferably a mixture of C₈-C₁₈ polyglycoside and carboxylated C₈-C₁₈ polyglycoside, more preferably a mixture of C₁₀-C₁₆ polyglycoside and carboxylated C₁₀-C₁₆ polyglycoside, still more preferably a mixture of C₁₂-C₁₄ polyglycoside and carboxylated C₁₂-C₁₄ polyglycoside.

In some embodiments of the present invention, the oral care composition comprises one N-acyl amino acid surfactant as described hereinabove and one alkyl polyglycoside as described hereinabove. In some embodiments of the present invention, the oral care composition comprises one N-acyl amino acid surfactant as described hereinabove and one carboxylated alkyl polyglycoside as described hereinabove. In some embodiments of the present invention, the oral care composition comprises N-acyl amino acid surfactant as described hereinabove, alkyl polyglycoside as described hereinabove and carboxylated alkyl polyglycoside as described hereinabove.

In some preferred embodiment, the oral care composition comprises N-acyl glutamic acid and its salts thereof and C₈-C₁₈ alkyl polyglycoside, preferably C₁₀-C₁₆ alkyl polyglycoside. In some preferred embodiment, the oral care composition comprises N-acyl glutamic acid and its salts thereof, C₈-C₁₈ alkyl polyglycoside as described hereinabove and carboxylated C₈-C₁₈ alkyl polyglycoside as described hereinabove, preferably C₁₀-C₁₆ alkyl polyglycoside and carboxylated C₁₀-C₁₆ alkyl polyglycoside.

### Other Essential Agents

The oral care composition of the present invention may further comprise, in addition to the abovementioned surfactants, another surfactant or a mixture of compatible surfactants. Suitable surfactants are those which are reasonably stable throughout a wide pH range, i.e., non-soap anionic, cationic, nonionic or zwitterionic surfactants.

Suitable examples of anionic surfactants are: water-soluble salts of higher fatty acid monoglyceride monosulfates, such as the sodium salt of the monosulfated monoglyceride of hydrogenated coconut oil fatty acids; higher alkyl sulfates such as sodium lauryl sulfate; alkyl aryl sulfonates; higher alkyl sulfoacetates; and higher fatty acid esters of 1,2 dihydroxypropane sulfonate. Ampholytic surfactants, serving as anions, can also be included in the compositions of the present invention. Examples of such compounds are: sodium or potassium N-lauroyl sarcosine; ethanolamine salts of N-lauroyl, N-myristoyl, or N-palmitoyl sarcosine. Mixtures of anionic surfactants can be employed. Suitable examples of non-ionic surfactants are the copolymer of ethylene oxide and propylene oxide for example, poloxamer 407, which is sold under the tradename Pluracare® F-127 NF Prill by BASF, Poloxamer 338 (under the tradename of Puracare® F108 NF Prill), poloxamer 188 (under the tradename of Pluracare® F 68 G, PEG/PPG-116/66 Copolymer (under the tradename of Pluracare® L1220), polyethylene oxide condensates of alkyl phenols, products derived from the condensation of ethylene oxide with the reaction product of propylene oxide and ethylene diamine, and ethylene oxide condensates of aliphatic alcohols.

The oral care composition of the present invention may also include a solubilizing agent, or emulsifying agent, such as ethoxylated castor oil. Similarly, any known solubilizing agent suitable for oral applications may be employed. Other suitable solubilizing agents include, for example hydrogenated castor oil, or PEG 40 or PEG 60 hydrogenated castor oil, and polysorbates. The solubilizing agent may be present from about 1.5 to about 5% by weight based on the total composition weight.

In addition to the above described essential components, the embodiments of this invention can contain a variety of optional oral care ingredients some of which are described below. Optional ingredients include, for example, but are not limited to, adhesives, sudsing agents, flavoring agents, sweetening agents, additional antiplaque agents, abrasives, and coloring agents.

The oral care composition of the present invention can be used in a variety of suitable oral vehicles. In this regard, anti-plaque benefits can be provided via a chewing gum, mouthwash, oral paste, gel, or powder. Any vehicle to which the delivery system of the present invention can be added can be used.

The oral care compositions of the present invention may further contain fluoride ions. Fluoride ions may be provided by a fluoride ion source. A fluoride ion source may be anything that is capable of releasing fluoride ion in an aqueous environment. Typical sources include soluble salts of the fluoride ion; such as, for example: sodium fluoride, potassium fluoride, calcium fluoride, zinc fluoride, zinc ammonium fluoride, lithium fluoride, (alkyl) ammonium fluoride, stannous fluoride, stannous fluorozirconate, and complex fluorides, monofluorophosphates and salts thereof such as, e.g. sodium monofluorophosphate or potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, and amine fluorides.

The fluoride ion source is most preferably in an amount such that it is capable of maintaining a high level of fluoride ion in the composition that is at least about 250 ppm, and in some instances up to as much as about 25,000 ppm. Preferably, the fluoride ions are present in an amount of about 1000 ppm to about 1500 ppm. In order to provide such a concentration in the optimal ppm range, the exact weight percentage of the fluoride ion source in the composition may vary, depending upon the stoichiometric properties of different fluoride ion sources.

The oral care compositions of the present invention may comprise a thickening agent, in particular for toothpaste composition. Suitable thickening agents are known in the art, and include, but are not limited to: silica thickeners; glycerites; carboxyvinyl polymers, carrageenan, gums such as tragacanth, ghatti, acacia, veegum; sodium alginate; carboxymethyl cellulose and its water-soluble salts; hydroxyethyl cellulose, hydroxypropyl cellulose; hydroxymethyl cellulose; hydroxymethyl carboxypropyl cellulose; methyl cellulose; ethyl cellulose; sulfated cellulose; xanthan gum, guar gum, hydroxylpropyl guar, gellan gum, carbomer and its salts; as well as mixtures and combinations of these compounds. Such additional thickeners may be used in amounts of up to about 15 wt % of the composition based on the total weight of the composition. In preparing toothpaste, it is necessary to add some thickening agent to provide a desirable consistency and the thickening agent is in an amount from 0% to 15% by weight of the total composition.

The oral care compositions of the present invention may also include a variety of common additional active ingredients/agents typically used in oral care formulations, including but not limited to: triclosan; triclosan monophosphate; chlorhexidine; alexidine; hexetidine; sanguinarine; benzalkonium chloride; salicylanilide; arginate esters; ethyl lauryl arginate, bisphenols, domiphen bromide; tetradecylpyridinium chloride; N-tetradecyl-4-ethylpyridinium chloride; octenidine; delmopinol; octapinol; nisin; zinc ion agent; copper ion agent; essential oils; furanones; bacteriocins; salts of the foregoing active ingredients/agents and mixtures and combinations thereof.

Optional additives for the oral care compositions of the present invention may also be used, such as those commonly used for forming a dentifrice, including but not limited to: abrasives and/or amorphous silica, humectants, stabilizing agents, antibacterial agents, sweeteners, colorants, healing agents, other caries preventative agents, chelating/sequestering agents, vitamins, amino acids, proteins, anti-plaque agents, anti-calculus agents, opacifiers, antibiotics, anti-enzymes, enzymes, pH control agents, oxidizing agents, antioxidants, whitening agents, basic amino acids (in free base or salt form) and preservatives.

Abrasive polishing materials might also be incorporated into the oral care compositions of the present invention. Suitable abrasives can be any material which does not excessively abrade dentin and does not provide calcium ions that may precipitate with, for example, the fluoride ions provided by any included fluoride ion source or that might complex with the composition's chelating agent. Suitable abrasives include, for example, silicas including gels and precipitates, insoluble sodium polymetaphosphate, beta-phase calcium pyrophosphate and resinous abrasive materials such as particulate condensation product of urea and formaldehyde. Combinations of abrasives may also be used.

Flavoring agents can also be added to oral dentifrice compositions of the present invention. Appropriate flavoring agents include oil of wintergreen, oil of peppermint, oil of spearmint, oil of sassafras, oil of clove and any other of the many known flavoring agents or combinations thereof.

Antibacterial agents can be used if reduction of microorganisms is desired, and can include known antibacterial agents used in dentifrice formulations such as, e.g., benzoic acid, sodium benzoate, potassium benzoate, boric acid, and phenolic compounds such as betanaphthol, chlorothymol, thymol, anethole, eucalyptol, carvacrol, menthol, phenol, amylphenol, hexylphenol, heptylphenol, octylphenol, hexylresorcinol, laurylpyridinium chloride, myristylpyridinium chloride, cetylpyridinium fluoride, cetylpyridinium chloride and cetylpyridinium bromide.

According to some embodiments of the present invention, the composition may further comprise active ingredients/agents for their anti-inflammatory properties, for example, Panthenol or pantothenate and a retinol or retinol derivative.

Suitable preservatives for use in the present invention include parabens (methyl and propyl parabens), sodium benzoate, benzyl alcohol and potassium sorbate.

Sweeteners may be used in the oral care compositions of the present invention if desired and may include any of those commonly used in a dentifrice to impart a pleasing taste to the product. Suitable sweeteners include but are not limited to: saccharins and derivatives thereof, cyclamates and derivatives thereof, acesulfane-K, thaumatin, neohisperidin dihydrochalcone, ammoniated glycyrrhizin, dextrose, levulose, sucrose, mannose, glucose and any other suitable sweeteners.

Preferred compositions include a humectant, e.g. xylitol, glycerol or sorbitol. Glycerol and sorbitol are particularly preferred. In some embodiments of the present invention, the toothpaste compositions include 5 to 70 wt % humectant. More preferred compositions include 15 to 55 wt % humectants while further preferred compositions include 30 to 45 wt % humectants. In some another preferred embodiment, the mouthwash or mouthrinse composition may comprise 0 to 55%, preferably from 2% to 40% or from 5 to 25% of humectant by weight based on the total weight of the composition.

In some preferred embodiment, the present composition may further comprise polyhydric alcohols which are best known for their solvent and humectant properties. These alcohols are soluble in water, alcohols, ethers and lower aliphatic hydrocarbons and also act to solubilize the flavoring agents of the present invention. The polyhydric alcohols useful in the present invention include those selected from the group consisting of propylene glycol, butylene glycol, pentylene glycol, hexylene glycol and mixture thereof. Preferably, the oral composition of the present invention comprises propylene glycol. The polyhydric alcohols comprise from 0% to 20% of the oral composition, preferably from 5% to 15%, or from 6% to 10%.

In the oral care compositions of the present invention, an adhesive is also desirable helping the active ingredients to adhere to the tissues of the oral cavity. Suitable adhesives include both polymers with limited water solubility as well as polymers lacking water solubility. These polymers deposit a thin film on both the oral cavity's soft and hard tissues when saliva combines with the instant composition. Suitable limited water solubility adhesives include: hydroxy ethyl or propyl cellulose. Adhesives lacking water solubility include ethyl cellulose, polyox resins and silicones. Adhesives lacking water solubility are incorporated into the instant invention by using a small amount of ethyl alcohol or another alcohol safe for use in the oral cavity and the human body. Another preferred adhesive suitable for being used in the present composition is polyvinylpyrrolidone (“PVP”) with a molecular weight of about 50,000 to about 300,000, which is available from BASF under the tradename of Luviskol?.

One person skilled in the art would recognize that an ingredient may serve various purposes in the composition. The other ingredients noted may be any of those commonly used in oral care formulations and can be selected based upon the intended end use of the formulations and to optimize the physical and aesthetic characteristics of the formulations.

The pH of the oral care composition may be greater than pH 3 to 10.5, preferably from 5.5 to 10, more preferably from 5.5 to 8. The oral care compositions herein may include an effective amount of a pH modifying agent, alternatively wherein the pH modifying agent is a pH buffering agent. pH modifying agent may include alkali metal hydroxides, ammonium hydroxide, organic ammonium compounds, carbonates, borates, silicates, phosphates, imidazole, citric acid, and sodium citrate.

In some preferred embodiments of the present invention, the oral care composition can be toothpaste. Water may be present in the toothpaste of this invention. Water, employed in the preparation of commercial suitable toothpaste should, preferably, be deionized and free of organic impurities. Water commonly comprises from about 10% to 50%, preferably from about 15% to about 40% and most preferably from about 20% to about 25% by weight of the oral care compositions described herein. This amount of water includes the free water which is added plus that amount which is introduced with other materials such as with sorbitol or any components of the invention.

In some preferred embodiments of the present invention, the oral care composition of the present invention can be a mouth rinse composition. The mouth rinse composition can comprise from 50% to 90%, preferably from 70% to 85% of the mouth rinse composition. These amounts of water include the free water which is added, plus that amount which is introduced with other materials such as with sorbitol. The water used in the present invention should preferably be deionized, distilled, flee of organic impurities and bacteria and substantially free of metal ions.

The oral care composition of the present invention may further comprise a cooling agent or a combination of cooling agents. Suitable cooling agents include but are not limited to N-ethyl-p-menthane-3-carboxamide, N,2,3-trimethyl-2-isopropylbutaneamide, and 3-1-menthoxypropane 1,2-diol.

The suitable oral carriers for such compositions of the present invention are preferably in the form of conventional oral care products, for example, a suspension (e.g., mouth rinse), a dentifrice (e.g., toothpaste, gel or tooth powder), chewing gum, chewable tablets, lozenge or sachet. The oral care composition is preferably in the form of a dentifrice. Preferred dentifrices include but are not limited to various types of toothpaste, tooth polish, tooth gel, tooth tablet, tooth powder, mouth spray, mouthwash and mouth rinse, denture adhesive or cream or the like, preferably toothpaste, mouth wash or mouth rinse.

The oral care composition of the present invention can result in unexpected benefits both in anti-plaque activity and in reduced staining of teeth. The use of either a biosurfactant or an anionic or nonionic surfactant alone results in a reduction or inhibition of plaque in dental surfaces. The composition of the present invention, where both the biosurfactant and the anionic or nonionic surfactant are used, generally result in a synergistic reduction or inhibition of plaque compared to the separate use of either compound alone.

The oral care composition of the present invention can be made using methods which are common in the oral products area by the skilled person in the art.

The present invention also provides a method for removing plaque present on dental surfaces or preventing plaque, comprising the application of a safe and effective amount of the oral care composition of the present invention to the teeth and other oral surfaces.

The present invention also provides use of the oral care composition of the present invention for removing plaque present on dental surfaces or preventing plaque.

The following examples further describe and demonstrate preferred embodiments within the scope of the present invention. The examples are given solely for illustration and are not to be construed as limitations of this Invention as many variations are possible without departing from the spirit and scope thereof.

### EXAMPLES

### Materials

Plantapon® LGC sorb (referred to “LGC” in the below formulation table): sodium lauryl glucose carboxylate (15-25%), lauryl glucoside (10-20%) and water (60-70%), from BASF
Plantacare® 1200 UP (referred to “APG” in the below formulation table): Lauryl Glucoside (50-60%), Water (40-50%), from BASF
Plantapon® ACG 50 (referred to “ACG” in the below formulation table): Sodium Cocoyl Glutamate (40-50%), Propylene Glycol (4-7%), Water (43.5-45.5%), from BASF
Texapon® OC-N (referred to “SLS” in the below formulation table): Sodium Lauryl Sulfate (100%), from BASF
Pluracare® E 600 (referred to “PEG-12” in the below formulation table): PEG-12 (100%), from BASF
Dehyton PK 45 (referred to “CAPB” in the below formulation table): Cocamidopropyl Betaine (44-46%), Water (54-56%), from BASF
BioToLife™: Sophorolipid (Yeast Ferment Extract 30-50%), Water (50-70%), from BASF
Pluracare® F 127 NF Prill (referred to “Poloxamer 407” in the below formulation table): EO-PO-EO copolymer (100%), from BASF
Luviskol® K 30 Powder: Polyvinylpyrrolidone (100%), from BASF
Flavor (also referred to Flavor in the below formulation table): Spearmint oil flavoring, Optaminte from Symris.
Sodium Saccharin: from Shanghai Fortunechemical Co., Ltd.

The formulations of the examples and the comparative examples are listed in below table.

### Formulations

| | Ingredients/weight % (active matter) | | | | | | |
|---|---|---|---|---|---|---|---|
| | A0 | A1 | A2 | A3 | A4 | A5 | A6 |
| Water | To 100 | To 100 | To 100 | To 100 | To 100 | To 100 | To 100 |
| Sodium Saccharin | 0.05 | 0.05 | 0.05 | 0.05 | 0.05 | 0.05 | 0.05 |
| Panthenol | 0.5 | 0.5 | 0.5 | 0.5 | 0.5 | 0.5 | 0.5 |
| Glycerin | 10.0 | 10.0 | 10.0 | 10.0 | 10.0 | 10.0 | 10.0 |
| Sorbitol | 5.5 | 5.5 | 5.5 | 5.5 | 5.5 | 5.5 | 5.5 |
| Propylene Glycol | 6.0 | 6.0 | 6.0 | 6.0 | 6.0 | 6.0 | 6.0 |
| Polyvinylpyrrolidone | 0.5 | 0.5 | 0.5 | 0.5 | 0.5 | 0.5 | 0.5 |
| Sodium Hydroxide | q.s. | q.s. | q.s. | q.s. | q.s. | q.s. | q.s. |
| Flavor | 0.3 | 0.3 | 0.3 | 0.3 | 0.3 | 0.3 | 0.3 |
| Poloxamer 407 | 0.5 | 0.5 | 0.5 | 0.5 | 0.5 | 0.5 | 0.5 |
| Sophorolipid | — | — | — | — | — | — | — |
| Surfactant type | — | LGC | APG | ACG | SLS | PEG-12 | CAPB |
| Surfactant weight (%) | — | 0.2 | 0.2 | 0.2 | 0.2 | 0.2 | 0.2 |
| Benzyl Alcohol | 0.50 | 0.50 | 0.50 | 0.50 | 0.50 | 0.50 | 0.50 |
| Citric Acid | q.s. | q.s. | q.s. | q.s. | q.s. | q.s. | q.s. |
| pH | 6.5-7 | | | | | | |
| | Ingredients/weight % (active matter) | | | | | | |
| | B0 | B1 | B2 | B3 | B4 | B5 | B6 |
| Water | To 100 | To 100 | To 100 | To 100 | To 100 | To 100 | To 100 |
| Sodium Saccharin | 0.05 | 0.05 | 0.05 | 0.05 | 0.05 | 0.05 | 0.05 |
| Panthenol | 0.5 | 0.5 | 0.5 | 0.5 | 0.5 | 0.5 | 0.5 |
| Glycerin | 10.0 | 10.0 | 10.0 | 10.0 | 10.0 | 10.0 | 10.0 |
| Sorbitol | 5.5 | 5.5 | 5.5 | 5.5 | 5.5 | 5.5 | 5.5 |
| Propylene Glycol | 6.0 | 6.0 | 6.0 | 6.0 | 6.0 | 6.0 | 6.0 |
| Polyvinylpyrrolidone | 0.5 | 0.5 | 0.5 | 0.5 | 0.5 | 0.5 | 0.5 |
| Sodium Hydroxide | q.s. | q.s. | q.s. | q.s. | q.s. | q.s. | q.s. |
| Flavor | 0.3 | 0.3 | 0.3 | 0.3 | 0.3 | 0.3 | 0.3 |
| Poloxamer 407 | 0.5 | 0.5 | 0.5 | 0.5 | 0.5 | 0.5 | 0.5 |
| Sophorolipid | 0.5 | 0.5 | 0.5 | 0.5 | 0.5 | 0.5 | 0.5 |
| Surfactant type | — | LGC | APG | ACG | SLS | PEG-12 | CAPB |
| Surfactant weight (%) | — | 0.2 | 0.2 | 0.2 | 0.2 | 0.2 | 0.2 |
| Benzyl Alcohol | 0.50 | 0.50 | 0.50 | 0.50 | 0.50 | 0.50 | 0.50 |
| Citric Acid | q.s. | q.s. | q.s. | q.s. | q.s. | q.s. | q.s. |
| pH | | | | | | | |

The weight % based on the active matter.
The term “q.s.” is an abbreviation of the Latin phrase quantum sufficiat which means as much as needed.
The amounts of Sodium Hydroxide and Citric acid were “q.s.” to adjust the pH to 6.5-7.

### Measurements

### 1. Dental Plaque Removal Efficacy Evaluation

Dental plaque removal efficacy has been verified and evaluated in panelists. Several panelists used each oral care example composition and had baseline photographs taken of their teeth prior to using each product (T0). The plaque disclosing solution (from Tampei Pharmaceutical Co., Ltd) to dye the teeth bacterial film (dental plaque) was applied and a picture was taken to record the dyed area (T1). Water was used to rinse for 6 seconds to remove the dye not being attached to the dental plaque and a picture was taken to record the dyed area (T2). The oral care example formulations were used to rinse for 10 seconds and a picture was taken to record the dyed area (T3). Water was used then to rinse for 10 seconds and a picture was taken to record the dyed area (T4). The teeth of the panelists were scored for plaque area (dyed plaque area). Photographs of the panelists' teeth were converted to a digital image in a computer. The dyed tooth areas of each panelist identified to the computer were converted into numerical units of area and the units totaled.

The pigment for dyeing dental plaque is a commercialized plaque indicator in red color. The redder areas appeared; the more plaque left on the teeth.

$$The plaque removal efficacy = [ T 2 ( dyed tooth area \% before treatment ) -$$

$$T 4 ( dyed tooth area \% after treatment ) ] /$$

$$T 2 ( dye tooth area \% before treatment )$$

### Results:

Table 1a shows the dental plaque removal efficacy with the oral care compositions not comprising sophorolipid.

TABLE 1a

| | A0 | | | | | | |
|---|---|---|---|---|---|---|---|
| Samples | (Blank) | A1 | A2 | A3 | A4 | A5 | A6 |
| Plaque Removal | 50.3% | 58.3% | 72.7% | 46.7% | 99.7% | 35.3% | 89.4% |
| Efficacy | | | | | | | |
| Efficacy | 0.0% | 8.0% | 22.4% | −3.6% | 49.4% | −15.0% | 39.1% |
| Improvement* | | | | | | | |

*Efficacy improvement is obtained by subtracting the blank (control) sample.

Table 1b shows the dental plaque removal efficacy with the oral care compositions comprising sophorolipid and at least one surfactant.

TABLE 1b

| Samples | B0 | B1 | B2 | B3 | B4 | B5 | B6 |
|---|---|---|---|---|---|---|---|
| Plaque | 59.9% | 77.1% | 98.7% | 67.4% | 90.8% | 45.5% | 93.7% |
| Removal | | | | | | | |
| Efficacy | | | | | | | |
| Efficacy | 9.6% | 26.8% | 48.4% | 17.1% | 40.5% | −4.8% | 43.4% |
| Improvement* | | | | | | | |

*Efficacy improvement is obtained by subtracting the blank (control) sample.

The results shows that the combinations of sophorolipid and at least one surfactant selected from the group consisting of alkyl polyglycoside surfactant, carboxylated alkyl polyglycoside surfactant and N-acyl amino acid surfactant have synergy effect in dental plaque removal and prevention compared to the separate use of either compound alone.

### 2. Oral Irritation Evaluation

The oral irritation evaluation was conducted to test the surfactant mildness and oral irritation. The test sample solution was prepared in aqueous solution with 1% active content at pH of 7. The test sample solutions were applied to the oral tissue cells based on EpiOral Tissue Model provided by Mattek Corp. The viability of the cells over time was measured and used to calculate the time of exposure until viability is reduced to 50%, which is indicated by ET-50.

The oral irritation levels are classified as below.
ET-50>240 minutes, none to minimal irritation minutes>ET-50>150 minutes, Mild irritation 150 minutes>ET-50>10 minutes, moderate irritation ET-50<10 minutes, Severe irritation

Table 2 shows the oral irritation tests of the surfactants and biosurfactants in EpiOral Tissue Model.

| | ET-50 based on EpiOral | | |
|---|---|---|---|
| Samples | Model | | Oral Irritation Classification |
| BioToLife ™ (Sophorolipid) | >240 | min | None to minimal irritation |
| Plantacare ® 1200 UP | >240 | min | None to minimal irritation |
| Plantapon ® LGC Sorb | >240 | min | None to minimal irritation |
| Plantapon ® ACG 50 | >240 | min | None to minimal irritation |
| Texapon ® OC-N | 116.7 | min | Moderately irritation |
| Pluracare ® E-600 | Not available | | None to minimal irritation |
| Dehyton ® PK 45 | 181.0 | min | Mildly irritation |

The results show that the surfactants used in the inventive examples not only demonstrating the synergy effect in dental plaque removal with sophorolipid but also have the advantages of mildness and no or just minimal irritation, thus improve the mildness and reduce the irritation of the oral care composition.

1. An oral care composition comprising-at least
(i) a glycolipid, and
(ii) a surfactant selected from the group consisting of an alkyl polyglycoside surfactant, a carboxylate alkyl polyglycoside surfactant, an N-acyl amino acid surfactant, and a mixture thereof.

2. The oral care composition according to claim 13, wherein the sophorolipid is a mixture of acidic form and lactone form, and wherein about 10 to about 90 wt. % of the sophorolipid being in acidic form and the remainder of the sophorolipid being in the lactone form.

3. The oral care composition according to claim 1, where in the N-acyl amino acid surfactant is selected from the group consisting of an N—(C₆-C₂₀-acyl)amino acid and their salts, an N—(C₈-C₁₈-acyl)amino acid and their salts, and an N—(C₁₀-C₁₆-acyl)amino acid and their salts.

4. The oral care composition according to claim 1, wherein the N-acyl amino acid surfactant is selected from the group consisting of an N-acyl glutamic acid and its salts thereof, an N-acyl sarcosine and its salts thereof, an N-acyl taurine and its salts thereof, and a mixture thereof.

5. The oral care composition according to claim 1, wherein the alkyl polyglycoside surfactant has a formula (I):
R₁—O—(R₂—O)ₐ—(Z)_{b}—H (I)
wherein R₁ is a straight chain or branched C₆-C₂₄-hydrocarbyl,
R₂ is a straight chain C₂-C₄-alkylene,
a is a number in the range of 0 to 10,
Z is a residue of a reducing sugar having 5 to 6 carbons, and
b is a number in the range of 1 to 5, and any combination thereof.

6. The oral care composition according to claim 1, wherein the carboxylate alkyl polyglycoside surfactant has a formula (II):
R₃—O—(R₄—O)ₘ—(S)ₙ—CH₂COO⁻X⁺
wherein R₃ is a straight chain or branched C₆-C₂₄-hydrocarbyl,
R₄ is straight chain C₂-C₄-alkylene,
m is a number in the range of 0 to 10,
S is a residue of a reducing sugar having 5 to 6 carbons,
n is a number in the range of 1 to 5,
X is an alkali metal ion, and
any combination thereof.

7. The oral care composition according to claim 1, wherein the surfactant is present in an amount of more than 0 to 5% by weight based on the total weight of the composition.

8. The oral care composition according to claim 1, wherein the glycolipid is present in an amount of more than 0 to 10% by weight based on the total weight of the composition.

9. The oral care composition according to claim 1, further comprising one or more active ingredient and a suitable oral carrier.

10. The oral care composition according to claim 1 in the form of a toothpaste, tooth polish, tooth gel, tooth tablet, tooth powder, mouth spray, mouthwash, mouth rinse, or denture adhesive or cream.

11. A method for removing plaque present on a dental surface or preventing plaque, comprising applying a safe and effective amount of a composition according to claim 1, to teeth and other oral surfaces.

12. (canceled)

13. The oral care composition according to claim 1, wherein the glycolipid is a sophorolipid.
